(19) United States
(12) Patent Application Publication
(10) Pub. No.: US 2024/0100165 A1
(43) Pub. Date: Mar. 28, 2024

(54) SOLID SNEDDS BASED ON A SPECIFIC MIXTURE OF ACRYLIC POLYMERS

(71) Applicant: Evonik Operations GmbH, Essen (DE)

(72) Inventors: Christian Moers, Langen (DE); Fabian-Pascal Schmied, Langen (DE); Manuel Seibel, Bensheim (DE); Alexander Bernhardt, Nieder-Olm (DE)

(73) Assignee: Evonik Operations GmbH, Essen (DE)

(21) Appl. No.: 18/548,680

(22) PCT Filed: Feb. 24, 2022
(86) PCT No.: PCT/EP2022/054690

§ 371 Date: Sep. 1, 2023

(30) Foreign Application Priority Data
Mar. 4, 2021 (EP) 21160593.6

(51) Int. Cl. A61K 47/32; A61K 9/00; A61K 9/107

(52) U.S. Cl. CPC A61K 47/32; A61K 9/0053; A61K 9/1075

(57) ABSTRACT

A method of preparing a specific solid self-nanoemulsifying drug delivery system involves applying an obtained self-nanoemulsifying drug delivery system on a mixture. The mixture contains (ia) 60 to 90 parts by weight of at least one dimethylaminoethyl methacrylate-butyl methacrylate-methyl methacrylate copolymer; (iia) 10 to 40 parts by weight of at least one methacrylic acid-ethyl acrylate copolymer; and (iiia) optionally, at least one additive; where the sum of (a) and (iia) is 100 parts by weight. The solid self-nanoemulsifying drug delivery system obtained by the method of the present invention is useful as a medicament.

### CROSS-REFERENCE TO RELATED APPLICATIONS

This application is the National Stage entry under § 371 of International Application No. PCT/EP2022/054690, filed on Feb. 24, 2022, and which claims the benefit of priority to European Patent Application No. 21160593.6 filed on Mar. 4, 2021. The content of each of these applications is hereby incorporated by reference in its entirety.

### FIELD OF THE INVENTION

The present invention refers to a method of preparing a specific solid self-nanoemulsifying drug delivery system, which comprises applying the obtained self-nanoemulsifying drug delivery system on a mixture comprising or consisting of
(ia) 60 to 90 parts by weight of at least one dimethylaminoethyl methacrylate-butyl methacrylate-methyl methacrylate copolymer;
(iia) 10 to 40 parts by weight of at least one methacrylic acid-ethyl acrylate copolymer; and
(iiia) optionally at least one additive; wherein
the sum of (ia) and (iia) is 100 parts by weight. Furthermore, the present invention refers to the solid self-nanoemulsifying drug delivery system obtained by the method of the present invention and this system for use as a medicament.

### BACKGROUND

Self-nanoemulsifying drug delivery systems (SNEDDS) are well known in the field of pharmaceutical compositions. The systems use the emulsion of poorly water-soluble active ingredients to improve drug solubilization. In general, two kinds of SNEDDS are known, liquid or semi-liquid SNEDDS and solid SNEDDS (S-SNEDDS). While the liquid or semi-liquid character of SNEDDS is often seen as a disadvantage in view of dosing for peroral applications and in view of their lack of storage stability, solid self-nanoemulsifying systems are preferred.

General methods of providing solid self-nanoemulsifying drug delivery systems or in the neighboring field of self-microemulsifying drug delivery systems (SMEDDS) are for example disclosed in:

EP 2 101 729 B1, which describes in this regard several ways for the conversion of microemulsions into the solid state. These are adsorption on colloidal silicon dioxide, stabilization of individual phases with colloidal silicon dioxide and hydrophobic colloidal silicon dioxide, incorporation in polyethylene glycol dispersions and spray drying.

Silva, D. Luis Antonio, et al. (International Journal of Pharmaceutics 541 (2018) 1-10), which describes a preparation of a solid self-microemulsifying drug delivery system (S-SMEDDS) by hot melt extrusion. S-SMEDDS were prepared by blending carvedilol and a lipid mixture with hydroxyl propyl methyl cellulose acetate succinate (HPMCAS). Extrudates prepared at the lowest drug concentration and highest temperature and recirculation time promoted a complete and rapid drug release at pH 6.8.

CN107308133 A, which describes S-SMEDDS comprising curcumin containing SMEDDS employing AEROSIL® 200 as adsorbent.

The object of the present invention was to provide a pharmaceutical composition based on dimethylaminoethyl methacrylate-butyl methacrylate-methyl methacrylate copolymers which can provide good stability and fast drug release characteristics.

In this regard the inventors of the present invention surprisingly found that pharmaceutical compositions comprising
(ia) 60 to 90 parts by weight of at least one dimethylaminoethyl methacrylate-butyl methacrylate-methyl methacrylate copolymer;
(iia) 10 to 40 parts by weight of at least one methacrylic acid-ethyl acrylate copolymer; wherein the sum of (ia) and (iia) is 100 parts by weight, as base polymer mixture can solve the object.

Furthermore, it has been found that this specific copolymer combination is suitable for an “in situ” generation of a solid-SNEDDS composition, i.e. can be used in a method which requires only a single extrusion step. Additionally, it has been found that this combination is suitable for poorly soluble active ingredients.

### SUMMARY OF THE INVENTION

Therefore, in a first aspect, the present invention refers to a method of preparing a solid self-nanoemulsifying drug delivery system comprising or consisting of the steps:
providing a self-nanoemulsifying drug delivery system by mixing
(i) at least one pharmaceutically active ingredient;
(ii) at least one lipid component;
(iii) at least one surfactant;
(iv) optionally at least one solvent; and
(v) optionally at least one additive; and then
applying the obtained self-nanoemulsifying drug delivery system on a mixture comprising or consisting of
(ia) 60 to 90 parts by weight of at least one dimethylaminoethyl methacrylate-butyl methacrylate-methyl methacrylate copolymer;
(iia) 10 to 40 parts by weight of at least one methacrylic acid-ethyl acrylate copolymer; and
(iiia) optionally at least one additive; wherein
the sum of (ia) and (iia) is 100 parts by weight;
by hot melt extrusion, spray drying, adsorption, electrospinning, electrospraying, prilling by vibration, granulation or supercritical fluidization to obtain the solid self-nanoemulsifying drug delivery system.

In a second aspect, the present invention pertains to a solid self-nanoemulsifying drug delivery system obtained by the method of the present invention.

Finally, in a third aspect, the present invention refers to a solid self-nanoemulsifying drug delivery system according to the present invention for use as a medicament or nutraceutical product.

### DESCRIPTION OF THE FIGURES

FIG. 1: Dissolution profiles of S-SNEDDS and ASD incorporating fenofibrate and fenofibrate drug substance in 500 ml 0.1N HCl in USP apparatus II. Each value designates the mean±S.D. (n=3).

### DETAILED DESCRIPTION

The present invention refers to a method of preparing a solid self-nanoemulsifying drug delivery system comprising or consisting of the steps:
providing a self-nanoemulsifying drug delivery system by mixing
(i) at least one pharmaceutically active ingredient;
(ii) at least one lipid component;
(iii) at least one surfactant;
(iv) optionally at least one solvent; and
(v) optionally at least one additive; and then applying the obtained self-nanoemulsifying drug delivery system on a mixture comprising or consisting of
(ia) 60 to 90, preferably 70 to 80, parts by weight of at least one dimethylaminoethyl methacrylate-butyl methacrylate-methyl methacrylate copolymer;
(iia) 10 to 40, preferably 20 to 30, parts by weight of at least one methacrylic acid-ethyl acrylate copolymer; and
(iiia) optionally at least one additive; wherein the sum of (ia) and (iia) is 100 parts by weight;
by hot melt extrusion, spray drying, adsorption, electrospinning, electrospraying, prilling by vibration, granulation or supercritical fluidization, preferably by hot melt extrusion, more preferably by hot melt extrusion at 130 to 180° C., most preferably at 130 to 160° C., to obtain the solid self-nanoemulsifying drug delivery system.

Furthermore, the present invention refers to a solid self-nanoemulsifying drug delivery system obtained by the method of the present invention.

Finally, the present invention refers to a solid self-nanoemulsifying drug delivery system according to the present invention for use as a medicament.

These and other aspects, embodiments, features, and advantages of the invention will become apparent to a person skilled in the art through the study of the following detailed description and claims. Any feature from one aspect of the invention can be used in any other aspect of the invention.

Furthermore, it will readily be understood that the examples contained herein are intended to describe and illustrate the invention but not to limit the invention and that, in particular, the invention is not limited to these examples.

Numerical ranges that are indicated in the format “from x to y” also include the stated values. If several preferred numerical ranges are indicated in this format, it is self-evident that all ranges that result from the combination of the various endpoints are also included.

“One or more”, as used herein, relates to at least one and comprises 1, 2, 3, 4, 5, 6, 7, 8, 9 or more of the referenced species. Similarly, “at least one” means one or more, i.e. 1, 2, 3, 4, 5, 6, 7, 8, 9 or more. “At least one”, as used herein in relation to any component, refers to the number of chemically different molecules, i.e. to the number of different types of the referenced species, but not to the total number of molecules. For example, “at least one surfactant” means that at least one type of molecule falling within the definition for a surfactant is used but that also two or more different types of surfactants falling within this definition can be present, but does not mean that only one or more molecules of one type of surfactant are present.

All percentages given herein in relation to the compositions or formulations relate to wt.-% relative to the total weight of the respective composition, if not explicitly stated otherwise.

“Essentially free of” according to the present invention with regard to compounds means that the compound can only be present in an amount, which does not influence the characteristics of the composition, in particular the respective compound is present in less than 3 wt.-%, preferably 1 wt.-%, more preferably 0.01 wt.-%, based on the total weight of the composition or is not present at all.

The weight average molecular weight Mw and the number average molecular weight Mn can be determined by GPC employing polystyrene standards or SEC. For neutral or anionic (meth)acrylate (co)polymers the method described in M. Adler et. al. “Molar mass characterization of hydrophilic copolymers, 1 Size exclusion chromatography of neutral and anionic (meth)acrylate copolymers” e-Polymers, vol. 4, no. 1, 2004 can be used. For cationic (meth)acrylate (co)polymers the method described in N. Adler et. al. “Molar mass characterization of hydrophilic copolymers, 2 Size exclusion chromatography of cationic (meth)acrylate copolymers” e-Polymers, vol. 5, no. 1, 2005 can be used.

The glass transition temperature Tg may be determined by DSC (Differential Scanning Calorimetry) analysis according to DIN EN ISO 11357-2:2013 (measurement without addition of plasticizer at a residual monomer content (ReMo) of less than 100 ppm, heating rate 10° C./min, nitrogen atmosphere).

The median of the particle size's volume distribution D_{v,50} and Z-Average particle size D_{z} can be determined by dynamic light scattering (DLS) according to ISO 22412:2017 “Particle size analysis—Dynamic light scattering (DLS)”. The polydispersity index (PDI) is determined from a two-parameter fit to the correlation data (the cumulants analysis). The calculations used for the determination of PDI are defined in the ISO standard documents 22412:2017.

### Pharmaceutically Active Ingredient (i)

Any pharmaceutically active ingredient or mixtures of pharmaceutically active ingredients known to the skilled person may be incorporated in the pharmaceutical compositions and S-SNEDDS. However, the pharmaceutical compositions of the present invention are very useful for poorly water-soluble pharmaceutically active ingredients or for pharmaceutically active ingredients which show a high drug loss after storage. Preferably, the pharmaceutically active ingredient may be a drug poorly soluble, in particular water-soluble, after peroral administration.

The pharmaceutically active ingredient (i) may show a solubility of less than 0.1 mg of pharmaceutically active ingredient, preferably of the pure pharmaceutically active ingredient, in 1 ml water at 37° C. (as defined for poorly insoluble drugs in the USP). The determination of the solubility of the pharmaceutically active ingredient is well known to a person skilled in the art. For instance, an excess amount of the pharmaceutically active ingredient is placed in a certain amount of water and mixed. The dissolved amount of the pharmaceutically active ingredient is then determined by a suitable analytical method, for instance by spectrometry.

In one embodiment the at least one pharmaceutically active ingredient may be selected from acalabrutinib, albendazole, allendronic acid, aripiprazole, asenapine, atazanavir, atorvastatin, BETd-260 bleomycin, bosentan, BRD4 degrader AT1, buprenorphine, budesonide, camostat, candesartan, carbamazepine, carvedilol, celecoxib, cilazapril, clarithromycin, clodronic acid, clopidogrel, curcumin, cytarabine, darunavir, dasatinib, deferasirox, dexamethasone, dexlansoprazole, diclofenac, diltiazem, docetaxel, doxorubicin, duloxetine, dutasteride, efavirenz, elbasvir, eprosartan, erlotinib, estradiol, etidronic acid, etravirine, everolimus, ezetimibe, felodipine, fenofibrate, fluconazole, fluorouracil, foretinib-based PROTAC 7, glimepiride, grazoprevir, griseovulvin, hydrochlorothiazide, hydrocortisone, hydroxychloroquine, ibuprofen, imatinib, irbesartan, irinotecan, itraconazole, ivacaftor, ivermectin, ledipasvir, lamotrigine, linezolid, lisinopril, lopinavir, losartan, lumefantrine, mefloquine, mesalazine, methotrexate, metoprolol, modafinil, moexipril, morphine, mycophenolate, naloxone, nifedipine, nilotinib, nilvadipine, nitrendipine, olanzapine, olmesartan, omeprazole, ondansetron, paclitaxel, pamidronic acid, paracetamol, pemetrexed, perindopril, phenytoin, pibrentasvir, pioglitazone, prednisone, progesterone, quetiapine, raloxifene, raltegravir, ramipril, rebamipide, remdesivir, rilpivirine, risedronic acid, risperidone, ritonavir, rivaroxaban, rivastigmine, rosuvastatin, selegiline, sevelamer, sibutramine, sildenafil, simvastatin, sirolimus, sitagliptin, sofosbuvir, sorafenib, spirapril, sunitinib, tacrolimus, tadalafil, tamoxifen, telaprevir, telmisartan, tenoxicam, terbutaline, ticagrelor, tiludronic acid, trandolapril, troglitazone, umifenovir, valsartan, velpatasvir, vemurafenib, verapamil, ziprasidone, zoledronic acid and ZXH-3-26, or, where applicable, from pharmaceutically acceptable salt forms thereof or mixtures thereof.

In another embodiment, the at least one pharmaceutically active ingredient is selected from resveratrol from grape products or pro-anthocyanins or anthocyanins, in particular from bilberries or black currants, soluble dietary fiber products, such as psyllium seed, broccoli (sulphane), and soy or clover (isoflavonoids), flavonoids, alpha-linoleic acid from flax seed, beta-carotene from marigold petals.

Preferably, the at least one pharmaceutically active ingredient may be selected from celecoxib, efavirenz and fenofibrate or mixtures thereof.

### Lipid Component (ii)

Any lipid component which can be used for pharmaceutical compositions is in general suitable.

The at least one lipid component (ii) can be selected from medium chain triglycerides (C₆-C₁₂ fatty acids), long chain triglycerides (C₁₃-C₂₁ fatty acids), propylene glycol dicaprylate/dicaprate (Captex® 200), glyceryl tricaprylate/tricaprate (Captex® 300), glyceryl triricinoleate (Castor oil), medium chain triglycerides (lauric acid) (Coconut oil), glyceryl dibehenate (Compritol® 888 ATO), triglycerides (linoleic acid, oleic acid) (Corn oil), triglycerides (linoleic acid, oleic acid, palmitic acid) (Cottonseed oil), ethyl oleate (Crodamol™ EO), glyceryl tricaprylate/tricaprate (Crodamol™ GTCC), isopropyl myristate (IPM-100), glyceryl tricaprylate/tricaprate (Labrafac™ CC), glyceryl tricaprylate/tricaprate (Labrafac™ lipophil WL 1349), propylene glycol dicaprylate/dicaprate (Labrafac™ PG), long chain triglycerides/diglycerides/monoglycerides (monolinoleate) (Maisine® CC), glyceryl tricaprylate/tricaprate/trilaurate (Miglyol® 812), oleic acid (Pamolyn™ 100 Oleic Acid), triglycerides (oleic acid, palmitic acid) (olive oil), triglycerides (palmitic acid, oleic acid, linoleic acid), triglycerides (oleic acid, linoleic acid, palmitic acid), triglycerides (linoleic acid, oleic acid, palmitic acid) (palm oil), triglycerides (linoleic acid, oleic acid, alpha-linolenic acid, palmitic acid) (sesame oil), triglycerides (linoleic acid, oleic acid, stearic acid) (soybean oil), glyceryl triacetate (Kollisolv® GTA), glyceryl tricaprylate (Tricaprylin®), hard fat (triglycerides/diglycerides) and hard fat (triglycerides) (Witepsol® H 35) or any mixture thereof.

Expressions in the above list with brackets indicate the main components of the lipid component (example: medium chain triglycerides (lauric acid)). Expressions in the above list with slashes indicate the components of the lipid component (example: glyceryl tricaprylate/tricaprate/trilaurate).

Surfactant (iii)

Any surfactant which can be used for pharmaceutical compositions is in general suitable.

The at least one surfactant (iii), can comprise one or more surfactants and can be selected from polyoxyethylene (23) lauryl ether, polyoxyethylene (2) oleyl ether, glyceryl monooleate, medium chain monoglycerides/diglycerides (caprylate, caprate), glyceryl monocaprylate, propylene glycol monocaprylate, propylene glycol monocaprylate, polyoxyl-35 hydrogenated castor oil, polyoxyl-40 hydrogenated castor oil, lauroyl polyoxyl-32 glycerides, stearoyl polyoxyl-32 glycerides, polyoxyl-15 hydroxystearate, poloxamer 188 (triblock copolymer of polyoxyethylene and polyoxypropylene), poloxamer 407 (triblock copolymer of polyoxyethylene and polyoxypropylene), oleoyl polyoxyl-6 glycerides, linoleoyl polyoxyl-6 glycerides, lauroyl polyoxyl-6 glycerides, caprylocaproyl polyoxyl-8 glycerides, propylene glycol monolaurate (type II), propylene glycol monolaurate (type I), polyoxyl-40 stearate, diacetylated monoglyceride, glyceryl monooleate, polyglyceryl-3 dioleate, sorbitan monolaurate, sorbitan monooleate, sorbitan sesquioleate, sorbitan trioleate, glyceryl monostearate, d-alpha-tocopherol polyethylene glycol 1000 succinate (d-TPGS), polyoxyethylene sorbitan monolaurate, polyoxyethylene sorbitan monostearate and polyoxyethylene sorbitan monooleate or any mixture thereof.

### Solvent (iv)

A solvent can be employed in the present invention. Any solvent which can be used for pharmaceutical compositions is in general suitable.

The at least one solvent (iv) can be selected from diethylene glycol monoethyl ether, polyethylene glycol 200, polyethylene glycol 400, polyethylene glycol 6000, propane-1,2,3-triol, (z)-octadec-9-enylamine, polypropylene glycol, propylene glycol, 2-pyrrolidone and tetraethylene glycol or any mixture thereof.

In an embodiment, the composition is essentially free of solvent.

Additive (v) and (iiia)

Any additive which can be used for pharmaceutical compositions, different from (i) to (iv), is in general suitable. The at least one additive (v) and (iiia) can be the same or different. In one embodiment only (v) is present. In an alternative embodiment only (iiia) is present. In one embodiment essentially no additive is present.

Additives are preferably selected from antiadherents, like magnesium stearate; fillers, like lactose, mannitol, starches, cellulose and their derivatives; binders, like polyacrylates, starches, guar, xanthan, alginate, carrageenan, pectin, tragacanth, polysaccharides and their derivatives; flavors, like mint, cherry, anise, vanilla, raspberry; colors, like natural colorants, azo and xanthene compounds; pigments, like titanium dioxides, iron oxides, magnesium oxide; disintegrants, like starches, croscarmellose, crosslinked polyvinylpyrrolidone, sodium hydrogen carbonate preferably in combination with citric acid (for effervescent tablets); glidants, like silica gel, fumed silica, talc, magnesium carbonate, flow regulators, like highly dispersed silicon dioxide; antioxidants like vitamin A, vitamin E, vitamin C, retinyl palmitate, selenium, butylated hydroxyanisole, butylated hydroxytoluene; sweeteners, like sucrose, sorbitol, saccharin sodium, cyclamate, aspartame; and antistatics, like alkyl sulfonates or quaternary ammonium compounds preferably combined with polystyrene; or mixtures thereof.

### Mixture Comprising Copolymers and Optional Additive

In the present invention
a mixture comprising or consisting of
(ia) 60 to 90, preferably 70 to 80, parts by weight of at least one dimethylaminoethyl methacrylate-butyl methacrylate-methyl methacrylate copolymer;
(iia) 10 to 40, preferably 20 to 30, parts by weight of at least one methacrylic acid-ethyl acrylate copolymer; and
(iiia) optionally at least one additive; wherein
the sum of (ia) and (iia) is 100 parts by weight; is employed.

In general, every dimethylaminoethyl methacrylate-butyl methacrylate-methyl methacrylate copolymer is suitable, preferably the copolymer can be used in the field of pharmaceutical compositions. A person skilled in the field of methacrylic copolymers knows how to obtain such polymers, in particular by radical polymerization, preferably free radical polymerization. In a preferred embodiment the copolymer is obtained by a solution polymerization process.

In one embodiment the dimethylaminoethyl methacrylate-butyl methacrylate-methyl methacrylate copolymer is obtained by radically polymerizing the monomers dimethylaminoethyl methacrylate, butyl methacrylate, and methyl methacrylate in a ratio of
a) 30 to 70 wt.-%, preferably 45 to 55 wt.-%, dimethylaminoethyl methacrylate;
b) 15 to 35 wt. %, preferably 20 to 30 wt.-%, butyl methacrylate; and
c) 15 to 35 wt.-%, preferably 20 to 30 wt.-%, methyl methacrylate;
whereby the sum of a) to c) is 100 wt.-%; optionally in the presence of further additives.

Suitable further additives are at least one initiator, preferably selected from di-(3,5,5)trimethylhexanoyl peroxide, tert-butyl peroxyneodecanoate, tert-butyl perbenzoate, tert-amyl peroxy-2-ethylhexanoate, bisdecanoyl peroxide, tert-butyl peroxy-2-ethylhexanoate, tert-butyl peroxy-2-ethylhexylcabonate, 1,1′-azobis(cyclohexanecarbonitrile), benzoyl peroxide, 2,2-di-(tert-butylperoxy)butane, dicumyl peroxide, di-tert-amyl peroxide, di-tert-butyl peroxide, lauroyl peroxide, tert-butylperoxy-3,5,5-trimethylhexanoate, 1,1-di-tert-butylperoxy-3,3,5-trimethylcyclohexane, 2-(1-cyan-1-methyl(ethyl)azocarboxamide, tert-butyl peroxyacetate, tert-butyl peroxypivalate or mixtures thereof, more preferably selected from tert-butyl peroxyneodecanoate and tert-butyl peroxypivalate or mixtures thereof;
and/or at least one chain-transfer agent preferably selected from carbon tetrachloride, carbon tetrabromide, bromotrichloromethane, 4-methylbenzenethiol, isooctyl 3-mercaptopropionate, pentaphenylethane, tert-nonyl mercaptan, 4,4′-thiobisbenzenethiol and n-dodecyl mercaptan, more preferably the chain-transfer agent is n-dodecyl mercaptan, and/or at least one solvent.

For the polymerization reaction, any solvent which is suitable for use in those reactions is generally suitable.

In one embodiment the at least one dimethylaminoethyl methacrylate-butyl methacrylate-methyl methacrylate copolymer has a weight average molecular weight MW of from 15,000 to 300,000 g/mol, preferably 50,000 to 250,000 g/mol, more preferably 100,000 to 200,000 g/mol, more preferably 150,000 to 190,000 g/mol.

In one embodiment the at least one dimethylaminoethyl methacrylate-butyl methacrylate-methyl methacrylate copolymer has a residual monomer content of not more than 0.5% for each monomer.

The total and individual residual monomer contents can be determined by High Pressure Liquid Chromatography (HPLC). The determination of the total and individual residual monomer contents by HPLC is well known to a skilled person.

In one embodiment, the at least one dimethylaminoethyl methacrylate-butyl methacrylate-methyl methacrylate copolymer has a polydispersity of from 2.0 to 5, preferably 3.5 to 4.5.

In one embodiment, the at least one dimethylaminoethyl methacrylate-butyl methacrylate-methyl methacrylate copolymer has a glass transition temperature T_{g} of from 20 to 60° C., preferably 35 to 50° C.

In one embodiment, the at least one dimethylaminoethyl methacrylate-butyl methacrylate-methyl methacrylate copolymer is essentially free of reactive groups, like epoxy groups. In one embodiment, the at least one dimethylaminoethyl methacrylate-butyl methacrylate-methyl methacrylate copolymer is not able to undergo further polymerization reactions.

In general, every methacrylic acid-ethyl acrylate copolymer is suitable.

In one embodiment the at least one methacrylic acid-ethyl acrylate copolymer is obtained by radically polymerizing the monomers methacrylic acid and ethyl acrylate in a ratio of
a) 35 to 60 wt.-%, preferably 45 to 55 wt.-%, methacrylic acid;
b) 40 to 65 wt.-%, preferably 45 to 55 wt.-% ethyl acrylate;
whereby the sum of a) and b) is 100 wt.-%; optionally in the presence of further additives.

Suitable further additives are at least one initiator, preferably selected from di-(3,5,5)trimethylhexanoyl peroxide, tert-butyl peroxyneodecanoate, tert-butyl perbenzoate, tert-amyl peroxy-2-ethylhexanoate, bisdecanoyl peroxide, tert-butyl peroxy-2-ethylhexanoate, tert-butyl peroxy-2-ethylhexylcabonate, 1,1′-azobis(cyclohexanecarbonitrile), benzoyl peroxide, 2,2-di-(tert-butylperoxy)butane, dicumyl peroxide, di-tert-amyl peroxide, di-tert-butyl peroxide, lauroyl peroxide, tert-butylperoxy-3,5,5-trimethylhexanoate, 1,1-di-tert-butylperoxy-3,3,5-trimethylcyclohexane, 2-(1-cyan-1-methyl(ethyl)azocarboxamide, tert-butyl peroxyacetate, tert-butyl peroxypivalate or mixtures thereof, more preferably selected from tert-butyl peroxyneodecanoate and tert-butyl peroxypivalate or mixtures thereof;
and/or at least one chain-transfer agent preferably selected from carbon tetrachloride, carbon tetrabromide, bromotrichloromethane, 4-methylbenzenethiol, isooctyl 3-mercaptopropionate, pentaphenylethane, tert-nonyl mercaptan, 4,4′-thiobisbenzenethiol and n-dodecyl mercaptan, more preferably the chain-transfer agent is n-dodecyl mercaptan, and/or at least one solvent.

For the polymerization reaction, any solvent which is suitable for use in those reactions is generally suitable.

In one embodiment the at least one methacrylic acid-ethyl acrylate copolymer has a weight average molecular weight M, of from 15,000 to 800,000 g/mol, preferably 50,000 to 550,000 g/mol, more preferably 100,000 to 350,000 g/mol, more preferably 150,000 to 300,000 g/mol.

In one embodiment, the at least one methacrylic acid-ethyl acrylate copolymer has a glass transition temperature T_{g} of from 70 to 130° C., preferably 80 to 115° C.

In one embodiment the at least one methacrylic acid-ethyl acrylate copolymer is obtained by an emulsion polymerization process with an optional subsequent drying step.

In one embodiment the at least one methacrylic acid-ethyl acrylate copolymer has a residual monomer content of not more than 0.5%, based on the sum of all monomers.

The total and individual residual monomer contents can be determined by High Pressure Liquid Chromatography (HPLC). The determination of the total and individual residual monomer contents by HPLC is well known to a skilled person.

The copolymers in the mixture can be used in powder form, preferably having an average particle size D_{v,50} in the range of from 1 to 1,000 μm, more preferably from 2 to 100 μm. The powder can be obtained by milling and grinding.

The copolymers can be present in the initial mixture in a pre-mixed powder form or can be coextruded. In one embodiment the coextrusion is performed at 140 to 165° C., preferably 150 to 160° C.

In one embodiment, the pre-mixed powder, preferably co-extruded, of the copolymers has a glass transition temperature T_{g} from 45 to 130° C., more preferably from 60 to 110° C., even more preferably from 70 to 105° C.

### Solid Self-Nanoemulsifying Drug Delivery Systems (S-SNEDDS)

A self-nanoemulsifying drug delivery system forms and remains a nanoemulsion in contact with water or gastrointestinal fluids. A skilled person in the field of nanoemulsions knows how to prepare the self-nanoemulsifying drug delivery system of the present invention with commonly known methods. The Z-Average size D_{z} (Z-Average particle size D_{z}) of the particles in the nanoemulsion may be between 1 and 1,000 nm, in many cases between 100 and 500 nm or from 10 to 100 nm.

The nanoemulsion formation may happen during manufacturing of a pharmaceutical composition or by a medical professional or in vivo. The formation happens, when the emulsifying components and the pharmaceutically active ingredient are added to an aqueous media, preferably water. In one embodiment, the formation is performed under stirring of the mixture and/or heating the mixture to 30 to 60° C., preferably 45 to 55° C., more preferably 50° C. Stirring and/or heating can improve the provision of a homogenous mixture.

The emulsifying components of the self-nanoemulsifying drug delivery systems are usually comprising, or are consisting of, a lipid component, at least one surfactant and optionally a solvent and/or optionally an additive, i.e. components (ii) to (v). A skilled person in the field knows how to select the components and adjust the amounts of the components to the pharmaceutically active ingredient to be delivered. Thus, emulsifying components (ii) to (v) are mixed with a pharmaceutically active ingredient (i) into a solution, which forms a self-nanoemulsifying drug delivery system (SNEDDS).

In one embodiment
(i) is present in 0.1 to 15 wt.-%;
(ii) is present in 5 to 40 wt.-%;
(iii) is present in 5 to 60 wt.-%;
(iv) is present in 0 to 50, preferably 10 to 50 wt.-%;
(v) is present in 0 to 25, preferably 0.1 to 5, wt.-%; based on the total weight of the self-nanoemulsifying drug delivery system.

The self-nanoemulsifying drug delivery system (SNEDDS) and a carrier, in the present invention the specific mixture of copolymers (ia) and (iia) and optionally an additive (iiia), form after processing, for instance by hot melt extrusion, spray drying, adsorption, electrospinning, electrospraying, prilling by vibration, granulation or supercritical fluid technology the solid self-nanoemulsifying drug delivery system (S-SNEDDS). In one preferred embodiment, the S-SNEDDS are obtained by hot melt extrusion. If an additive (iiia) is present, the additive is preferably compounded with the copolymers (ia) and (iia). In a preferred embodiment, the copolymers (ia) and (iia), as well as the optional additive (iiia) are in powder form in an alternative embodiment the components are coextruded before applied as a mixture. More preferably, they have an average particle size D_{z} in the range of from 1 to 1,000 μm, most preferably from 100 to 500 μm. The powders can be obtained by conventional milling and grinding.

Additives (v) and (iiia) can be the same or different. In one embodiment the at least one additive is selected from antiadherents; binders; flavors; pigments; disintegrants; glidants; flow regulators; antioxidants; sweeteners; and antistatics; or mixtures thereof. In one embodiment only an additive
(iiia) is present. In one embodiment the system is essentially free of additives.

In one embodiment the self-nanoemulsifying drug delivery system is present in 1 to 30 wt.-%, based on the total weight of the self-nanoemulsifying drug delivery system and the mixture.

For example, the S-SNEDDS of the present invention are obtained by hot melt extrusion at a temperature of 140 to 180° C., preferably of 145 to 170° C. In one embodiment, the components may be extruded in a twin-screw extruder. In one embodiment, the components may be extruded in a twin-screw extruder at a torque of about 30 to 100 Ncm. Preferably, the components may be extruded in a twin-screw extruder at a torque of about 45 to 85 Ncm. The extruded mass may leave the extruder in the form of a strand, which may be comminuted by grinding and milling to a powder product.

### Use as a Medicament

The solid self-nanoemulsifying drug delivery system (S-SNEDDS), of the present invention are suitable for use (method of use) as a medicament or nutraceutical product, which preferably enhances the solubility of the included pharmaceutically active ingredient compared to the pharmaceutically active ingredient alone in the treatment of a disease of a human or an animal subject.

The invention in particular pertains to:
1. Method of preparing a solid self-nanoemulsifying drug delivery system comprising or consisting of the steps:
   providing a self-nanoemulsifying drug delivery system by mixing
   (i) at least one pharmaceutically active ingredient;
   (ii) at least one lipid component;
   (iii) at least one surfactant;
   (iv) optionally at least one solvent; and
   (v) optionally at least one additive; and then
   applying the obtained self-nanoemulsifying drug delivery system on a mixture comprising or consisting of
   (ia) 60 to 90, preferably 70 to 80, parts by weight of at least one dimethylaminoethyl methacrylate-butyl methacrylate-methyl methacrylate copolymer;
   (iia) 10 to 40, preferably 20 to 30, parts by weight of at least one methacrylic acid-ethyl acrylate copolymer; and
   (iiia) optionally at least one additive; wherein
   the sum of (ia) and (iia) is 100 parts by weight;
   by hot melt extrusion, spray drying, adsorption, electrospinning, electrospraying, prilling by vibration, granulation or supercritical fluidization, preferably by hot melt extrusion, more preferably by hot melt extrusion at 130 to 180° C., most preferably at 130 to 160° C., to obtain the solid self-nanoemulsifying drug delivery system.
2. The method according to item 1, wherein (ia) and (iia) are present in a pre-mixed powder form.
3. The method according to item 1, wherein (ia) and (iia) and optionally (iiia) are coextruded before being applied as mixture.
4. The method according to any one of the preceding items, wherein the at least one dimethylaminoethyl methacrylate-butyl methacrylate-methyl methacrylate copolymer
   i) is obtained by radically polymerizing the monomers dimethylaminoethyl methacrylate, butyl methacrylate, and methyl methacrylate in a ratio of
      a) 30 to 70 wt.-% dimethylaminoethyl methacrylate;
      b) 15 to 35 wt. % butyl methacrylate; and
      c) 15 to 35 wt.-% methyl methacrylate;
      whereby the sum of a) to c) is 100 wt.-%; optionally in the presence of further additives, like catalysts or stabilizers;
   and/or
   ii) has a residual monomer content of not more than 0.5%, preferably not more than 0.3%, more preferably not more than 0.1% for each monomer; and/or
   iii) has a weight average molecular weight M, of 15,000 to 300,000 g/mol, more preferably of 20,000 to 200,000 g/mol even more preferably of 20,000 to 80,000 g/mol; and/or iv) is obtained by a solution polymerization process optionally further comprising a subsequent drying and optional comminuting step.
5. The method according to any one of the preceding items, wherein the at least one methacrylic acid-ethyl acrylate copolymer
   i) is obtained by radically polymerizing the monomers methacrylic acid and ethyl acrylate in a ratio of
      a) 35 to 60, preferably 40 to 55, more preferably 45 to 52, wt.-% methacrylic acid;
      b) 40 to 65, preferably 45 to 60, more preferably 48 to 55, wt.-% ethyl acrylate whereby the sum of a) and b) is 100 wt.-%; optionally in the presence of further additives, like catalysts or stabilizers; and/or
   ii) has a weight average molecular weight M. of 15,000 to 800,000 g/mol, preferably of 20,000 to 600,000 g/mol, more preferably of 50,000 to 500,000 g/mol, even more preferably 100,000 to 400,000 most preferably, 200,000 to 350,000 g/mol; and/or
   iii) is obtained by an emulsion polymerization process with an optional subsequent drying step; and/or
   iv) has a residual monomer content of not more than 0.5%, more preferably not more than 0.1% even more preferably not more than 0.01% based on the sum of all monomers.
6. The method according to any one of the preceding items, wherein the at least one pharmaceutically active ingredient has a solubility of less than 0.1 mg in 1 ml water at 37° C. and/or is selected from celecoxib, efavirenz and fenofibrate or mixtures thereof.
7. The method according to any one of the preceding items, wherein the at least one lipid component is selected from C₆-C₁₂ fatty acid triglycerides; C₁₃-C₂₁ fatty acid triglycerides; propylene glycol dicaprylate/dicaprate; glyceryl tricaprylate/tricaprate; glyceryl triricinoleate; lauric acid triglycerides; glyceryl dibehenate; linoleic acid and oleic acid triglycerides; linoleic acid, oleic acid, and palmitic acid triglycerides; ethyl oleate; isopropyl myristate; monolinoleate triglycerides/diglycerides/monoglycerides; glyceryl tricaprylate/tricaprate/trilaurate; oleic acid; oleic acid and palmitic acid triglycerides; palmitic acid, oleic acid, and linoleic acid triglycerides; oleic acid, linoleic acid, and palmitic acid triglycerides; linoleic acid, oleic acid, and palmitic acid triglycerides; linoleic acid, oleic acid, alpha-linolenic acid, and palmitic acid triglycerides; linoleic acid, oleic acid, and stearic acid triglyceride; glyceryl triacetate; glyceryl tricaprylate; hard fat or any mixtures thereof.
8. The method according to any one of the preceding items, wherein the at least one surfactant is selected from polyoxyethylene (23) lauryl ether; polyoxyethylene (2) oleyl ether; glyceryl monooleate; caprylate and caprate monoglycerides/diglycerides; glyceryl monocaprylate; propylene glycol monocaprylate; polyoxyl-35 hydrogenated castor oil; polyoxyl-40 hydrogenated castor oil; lauroyl polyoxyl-32 glycerides; stearoyl polyoxyl-32 glycerides; polyoxyl-15 hydroxystearate; triblock copolymer of polyoxyethylene and polyoxypropylene; oleoyl polyoxyl-6 glycerides; linoleoyl polyoxyl-6 glycerides; lauroyl polyoxyl-6 glycerides; caprylocaproyl polyoxyl-8 glycerides; propylene glycol monolaurate; polyoxyl-40 stearate; diacetylated monoglyceride; polyglyceryl-3 dioleate; sorbitan monolaurate; sorbitan monooleate; sorbitan sesquioleate; sorbitan trioleate; glyceryl monostearate; d-α-tocopherol polyethylene glycol 1000 succinate; polyoxyethylene sorbitan monolaurate; polyoxyethylene sorbitan monostearate; and polyoxyethylene sorbitan monooleate or any mixtures thereof.
9. The method according to any one of the preceding items, wherein the at least one solvent is selected from diethylene glycol monoethyl ether, polyethylene glycol 200, polyethylene glycol 400, polyethylene glycol 6000, propane-1,2,3-triol, (z)-octadec-9-enylamine, polypropylene glycol, propylene glycol, 2-pyrrolidone, tetraethylene glycol and diethylene glycol monoethyl ether or any mixtures thereof.
10. The method according to any one of the preceding items, wherein the at least one additive is selected from antiadherents; binders; flavors; pigments; disintegrants; glidants; flow regulators; antioxidants; sweeteners; and antistatics; or mixtures thereof.
11. The method according to any of the preceding items, wherein the self-nanoemulsifying drug delivery system is present in 1 to 30 wt.-%, based on the total weight of the self-nanoemulsifying drug delivery system and the mixture.
12. The method according to any of the preceding items, wherein
   (i) is present in 0.1 to 15 wt.-%;
   (ii) is present in 5 to 40 wt.-%;
   (iii) is present in 5 to 60 wt.-%;
   (iv) is present in 10 to 50 wt.-%;
   (v) is present in 0 to 25 wt.-%; based on the total weight of the self-nanoemulsifying drug delivery system.
13. Solid self-nanoemulsifying drug delivery system obtained by the method of any of items 1 to 12.
14. Solid self-nanoemulsifying drug delivery system according to item 13, wherein the solid self-nanoemulsifying drug delivery system is a nutraceutical product or medicament.
15. The solid self-nanoemulsifying drug delivery system according to item 13 for use as a medicament.

### EXAMPLES

### Materials & Methods

### Materials

Fenofibrate (propan-2-yl 2-[4-(4-chlorobenzoyl)phenoxy]-2-methylpropanoate) obtained from D.K. Pharma Chem PVT Ltd. (Maharashtra, India) was used as model compound. A co-extrudate (IPEC 75/25) of dimethylaminoethyl methacrylate-butyl methacrylate-methyl methacrylate copolymer (2:1:1) and methacrylic acid-ethyl acrylate copolymer (1:1) from Evonik Operations GmbH (Darmstadt, Germany). Dimethylaminoethyl methacrylate-butyl methacrylate-methyl methacrylate (EUDRAGIT® E PO) and methacrylic acid-ethyl acrylate (EUDRAGIT® L 100-55) are commercially available products from Evonik Operations GmbH (Darmstadt, Germany). Polyoxyethylene (80) sorbitan monooleate (Tween® 80), d-α-Tocopherol polyethylene glycol 1000 succinate (d-TPGS) and polyoxyethylene (23) lauryl ether (Brij® 35) were purchased from Sigma Aldrich (Steinheim, Germany). Medium-chain triglycerides (Miglyol® 812) was obtained from Caesar & Loretz GmbH (Hilden, Germany). All other chemicals were of analytical grade and purchased commercially.

### Methods

Preparation of the co-extrudate (IPEC 75/25) of dimethylaminoethyl methacrylate-butyl methacrylate-methyl methacrylate copolymer (2:1:1) and methacrylic acid-ethyl acrylate copolymer (1:1) by Hot-Melt Extrusion

For the preparation of co-extrudate of dimethylaminoethyl methacrylate-butyl methacrylate-methyl methacrylate copolymer (2:1:1) and methacrylic acid-ethyl acrylate copolymer (1:1) (IPEC 75/25) the copolymers dimethylaminoethyl methacrylate-butyl methacrylate-methyl methacrylate (EUDRAGIT® E PO) and methacrylic acid-ethyl acrylate (EUDRAGIT® L 100-55) were blended at a ratio of 75 wt.-% to 25 wt.-%. To guarantee a homogeneous distribution of both copolymers a Turbular mixer from WAB Group (Nidderau-Heldenbergen, Germany) was used for approximately 10 min. The copolymer blend was processed via hot-melt extrusion technology to receive the IPEC 75/25 using a co-rotating Three-Tec ZE 9 twin screw extruder from Three-Tec GmbH (Seon, Switzerland) that exhibited a parallel screw design. The hot-melt extrusion process was characterized by recording the applied screw speed (100 rpm), the torque (3.9-4.8 Nm) and the process temperature of the four different heating zones (50/75/130/155° C.). The continuously generated strand, leaving the extruder at its nozzle (nozzle diameter of 3.0 mm), cooled down while it was transported using a conveying belt and was finally chopped into a coarse granule. The coarse IPEC 75/25 was ground (mesh size: 0.25 mm) using an Ultra Centrifugal Mill ZM 200 from Retsch GmbH (Haan, Germany). Additionally, the IPEC 80/20 as well IPEC 70/30 were manufactured with the same procedure applying ratios of 80 wt.-% (EUDRAGIT® E PO) to 20 wt.-% (EUDRAGIT® L 100-55) and 70 wt.-% (EUDRAGIT® E PO) to 30 wt.-% (EUDRAGIT® L 100-55) respectively.

### Preparation of Solid-SNEDDS (S-SNEDDS) and Amorphous Solid Dispersion (ASD) Via Hot-Melt Extrusion

A blend in a stoichiometric ratio of the compounds 1 to 4 as well as the pharmaceutically active ingredient fenofibrate, in the following referred to as drug as well, (Table 1) was prepared by mixing these substances in a beaker glass under moderate magnetic stirring for approximately 30 min. The blend was subjected to a temperature of 50° C. while stirring. The obtained SNEDDS-solution (Table 1) was added to a certain polymer considering a defined mixture ratio. The solution/polymer blend (Table 2) was processed via hot-melt extrusion technology using a co-rotating Three-Tec ZE 9 twin screw extruder from Three-Tec GmbH (Seon, Switzerland) that exhibited a parallel screw design. The hot-melt extrusion process was characterized by recording the applied screw speed, the torque and the process temperature of the four different heating zones. The continuously generated strand, leaving the extruder at its nozzle (nozzle diameter of 3.0 mm), cooled down while it was transported using a conveying belt and was finally chopped into a coarse granule. The granule was ground (mesh size: 0.25 mm) using an Ultra Centrifugal Mill ZM 200 from Retsch GmbH (Haan, Germany). The obtained powder was the dosage form of the manufactured S-SNEDDS that was used for all further studies. In addition, a blend of polymer and drug substance (ASD) (Table 2) by using a Turbular mixer from WAB Group (Nidderau-Heldenbergen, Germany) for approximately 10 min was processed via the mentioned hot-melt extrusion process in order to assess the effect of S-SNEDDS referring to different characteristics in comparison to ASD. For ASD the copolymers IPEC 75/25 and EUDRAGIT® E PO were applied.

### “In Situ” Preparation of S-SNEDDS (IPEC 75/25 AIO (all in One) SNEDDS)

The mentioned SNEDDS solution (Table 1) was added to an unprocessed, dry blend of EUDRAGIT® E PO at 75 wt.-% and EUDRAGIT® L 100-55 at 25 wt.-% obtained by using a Turbular mixer from WAB Group (Nidderau-Heldenbergen, Germany) for approximately 10 min. For facilitating the hot-melt extrusion process of IPEC 75/25 AIO SNEDDS, the EUDGRAGIT® L 100-55 can be softened by mixing it with the SNEDDS solution first, within a maximum of 3 h. From this point on, the IPEC 75/25 AIO SNEDDS was manufactured by following the same procedure and equipment that was described for the method “Preparation of Solid-SNEDDS (S-SNEDDS) and amorphous solid dispersion (ASD) via hot-melt extrusion”.

### Rheological Measurement (DIN ISO 6721-10:2015)

For the rheological analysis of the IPECs a plate-plate measuring system with a diameter of 25 mm was used according to the complex shear modulus. In shear rheometry, the complex shear modulus described the behaviour of viscoelastic materials under oscillating shear stress. The complex viscosity of the IPECs (IPEC 80/20, IPEC 75/25 and IPEC 70/30) was measured in a selected a temperature range of 105-250° C. in a continuous process by applying a heating rate of 1° C./min. For graphical representation the complex viscosity n (Pa-s) was plotted against the temperature T (in ° C.). The rheological measurements were performed using an amplitude of 0.6% and an angular frequency of 10 rad/s. The determined temperature range, which covered a complex viscosity of 10³10⁴ Pa·s, was defined as suitable for a hot-melt extrusion process. The rheological measurements were conducted using the Modular Compact Rheometer MCR 302 from Anton Paar Germany GmbH (Bruchköbel, Germany).

### Dissolution Studies of S-SNEDDS, ASD and the Drug Substance Fenofibrate

Dissolution experiments were performed according to USP 42-NF 37 (2019). Dissolution experiments were conducted with 25 mg drug substance or an equivalent amount of S-SNEDDS or ASD using USP apparatus II (DT 800 LH) from ERWEKA GmbH (Langen, Germany). The paddle speed was set to 100 rpm and all experiments were performed in 500 ml of 0.1N hydrochloric acid. The dissolution tests were conducted over 120 min.

### HPLC Method for Analysing Fenofibrate

The high-performance liquid chromatography (HPLC) system (Agilent 1260 Infinity) was used for the quantification of fenofibrate consisted of a quaternary pump (G1311B), autosampler (G1329B), column oven (G1316A) and UV detector (G1314C), all obtained from Agilent Technologies (Frankfurt am Main, Germany). Separation was achieved using a Symmetry 300 C18 (150×4.6 mm, 5 μm) column maintained at 22° C. The mobile phase consisted of an acetonitrile: water mixture (70:30 v/v), adjusted to pH 2.50 with phosphoric acid. The flow rate was set to 2.0 ml/min. An injection volume of 20 μl was applied and fenofibrate was detected at 286 nm. In the concentration range of 0.14-595 μg/ml, the analytical curve was linear (r²=0.999996). The method was found to be accurate (101.2-102.1%) and precise (CV 2.78%) with a quantification limit of 0.05 μg/ml. Run time was defined to be 6 min. Selectivity was determined (formulation excipients) and no interference was observed in drug retention time. Moreover, the peak area did not change in the presence of all excipient used in the study.

### Differential Scanning Calorimetry (DSC) Analysis (DIN EN ISO 11357-2:2013)

The copolymers were thermally analysed via DSC to determine their glass transition temperature (T_{g}) and if the incorporated drug demonstrated an amorphous (glass transition) or crystalline (melting/crystallization peak) appearance. The glass transition is a reversible transition from a hard and relatively brittle, frozen state to a molten or rather rubbery state within amorphous or partly amorphous materials. The melting point of the pure drug substance as well as the glass transition temperature of the copolymers were investigated for identifying changes and/or shifts in the thermograms regarding crystalline and/or amorphous characteristics. A sample of 5-10 mg each was weighed into a small, perforated aluminum pan with a lid that was cold sealed and exposed to a heating-cooling-heating cycle starting from 0° C. up to 200° C. while running the measurement continuously applying inert nitrogen atmosphere. The constant heating/cooling rate was set to 10° C./min. In the resulting thermogram the heat flow is plotted against the temperature using an endothermic presentation method. The evaluation was based on the second heating cycle, and the indicated value is the mean value in the glass transition interval. The analysis was conducted using a DSC 3+(DSC-HC01) from Mettler Toledo (Gieβen, Germany).

### Stability Studies

S-SNEDDS and ASD were stored at constant and controlled conditions (30° C./65% RH) in a climatic chamber from Binder GmbH (Tuttlingen, Germany) over 6 months. The samples were kept in a 30 ml amber glass, closed with a screw cap. After 3 and 6 months, samples were withdrawn and the results regarding appearance, drug release and DSC were compared to the data of the samples at the time of manufacture.

### Fourier Transform-Infrared (FT-IR) Spectroscopy

The structural features of IPEC 75/25 as well as the copolymers EUDRAGIT® E PO and EUDRAGIT® L 100-55 were investigated by Fourier Transform-Infrared (FT-IR) spectroscopy in the range of 4000-400 cm⁻¹. The samples, approximately 10 mg each, were deposited on a diamond crystal of the FT-IR device, compacted by means of a metal attachment and measured subsequently. Valence and deformation vibrations could be detected by the FT-IR device after molecular excitation. Characteristic patterns (spikes) related to the chemical structure of the samples were identified by measuring the attenuated total reflection (ATR) of the exposed infrared radiation. The resulting FT-IR spectrogram was obtained by plotting the transmission [%] against the wave number [cm⁻¹]. The spectroscopy was conducted using the FT-IR spectrometer “ALPHA” from Bruker Optics (Hanau, Germany).

### Elementary Analysis of Nitrogen Content

The elements carbon (C), hydrogen (H) and nitrogen (N) bound in the test substances IPEC 75/25 and its physical powder mixture comprising the same percentage of EUDRAGIT® E PO and EUDRAGIT® L 100-55 as used in IPEC 75/25 were burned in a tin cartridge at approximately 1150° C. to the reaction products CO₂, H₂O, N₂ and NOₓ. The carrier gas flow (helium) transferred the gaseous combustion products into a reduction pipe, where the nitrogen oxides NOx were reduced to N₂. CO₂ and H₂O were adsorbed on the respective adsorption columns. The non-adsorbed N₂ entered the thermal conductivity detector as the first measuring component. After desorption, by heating out the adsorption columns, the remaining measuring components entered the measuring cell of the thermal conductivity detector with the carrier gas flow. Depending on the concentration of the measuring components, the thermal conductivity detector provided an electrical measurement signal. The analysis was conducted using the elementary analyser “Vario MICROcube” from Elementar Analysensysterne GmbH (Hanau, Germany).

### Results and Discussion

### Results

TABLE 1
Composition of SNEDDS-solution incorporating fenofibrate

| | compound 1 | compound | compound | compound | drug |
|---|---|---|---|---|---|
| | Miglyol ® | 2 | 3 | 4 | substance |
| trade name | 812 | Brij ® 35 | Tween ® 80 | d-TPGS | fenofibrate |
| amount [%] | 17.20 | 8.60 | 50.16 | 10.04 | 14.00 |

### Composition & Hot-Melt Extrusion Process Parameters of S-SNEDDS & ASD

The composition of the analysed samples including the SNEDDS load and/or drug load as well as the process parameters regarding the hot-melt extrusion process were recorded and presented in Table 2.

TABLE 2
Composition & hot-melt extrusion process parameters of S-SNEDDS and
ASD incorporating fenofibrate

| | total | total | extrusion | | screw |
|---|---|---|---|---|---|
| | SNEDDS | drug | temperature | torque | speed |
| sample name | load [%] | load [%] | zones [° C.] | [Nm] | [rpm] |
| IPEC 75/25 AIO | 30 | 4.2 | 125/135/145/155 | 2.7-3.7 | 100 |
| SNEDDS | | | | | |
| IPEC 75/25 | 30 | 4.2 | 125/135/145/155 | 3.0-3.5 | 100 |
| SNEDDS | | | | | |
| IPEC 75/25 (ASD) | 0 | 4.2 | 125/135/145/155 | 3.5-4.6 | 100 |
| EUDRDAGIT ® | 0 | 4.2 | 125/135/145/155 | 1.8-2.2 | 100 |
| E PO (ASD) | | | | | |

### Rheological Measurement

The extrusion temperature range for the different IPECs was determined according to the previously described method. IPECs with a higher percentage of EUDRAGIT® E PO demonstrated that they can already be processed at lower temperatures (Table 3).

TABLE 3
Extrusion temperature range for the different IPECs

| | extrusion temperature range |
|---|---|
| polymer | (10³ ≤ η ≤ 10⁴ Pa · s) [° C.] |
| IPEC 80/20 | 133-162 |
| IPEC 75/25 | 138-168 |
| IPEC 70/30 | 145-170 |

### Thermal Characterization of the Pure Polymers, S-SNEDDS & ASD Via DSC Analysis

All polymer samples (Table 4) were analysed via the mentioned DSC method to determine the T_{g} of the different polymers. The T_{g} of the manufactured IPECs became higher with increasing percentage of EUDRAGIT® L 100-55. All IPECs showed only one T_{g} in the temperature range that was specified. Table 5 shows the T_{g} of the samples processed via hot-melt extrusion process.

TABLE 4
Glass transition temperature (T_{g}) of the pure polymers

| polymer | T_{g} (polymer) [° C.] |
|---|---|
| EUDRAGIT ® E PO | 42 |
| EUDRAGIT ® L 100-55 | 96 |
| IPEC 80/20 | 79 |
| IPEC 75/25 | 88 |
| IPEC 70/30 | 94 |

TABLE 5
Glass transition temperature (T_{g}) of the pure polymers

| | | T_{g} (sample after |
|---|---|---|
| sample name | T_{g} (sample) [° C.] | 6 months) [° C.] |
| IPEC 75/25 AIO SNEDDS | 43 | N/D* |
| IPEC 75/25 SNEDDS | 45 | 43 |
| IPEC 75/25 (ASD) | 62 | 64 |
| EUDRDAGIT ® E PO (ASD) | 34 | N/D* |

*N/D = not determined

### Dissolution Studies

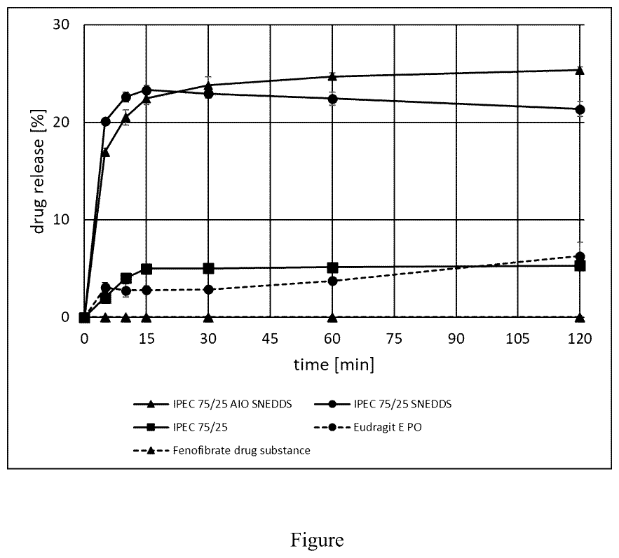

The highest, final level of drug release for the samples incorporating fenofibrate was achieved by IPEC 75/25 AIO SNEDDS closely followed by IPEC 75/25 SNEDDS (Table 6 and FIG. 1). The drug releases for the SNEDDS formulations were substantially higher compared to the ASDs. All samples were stable during the entire 120 min of the conducted dissolution test. After 6 months of storage the drug release just slightly decreases for IPEC 75/25 SNEDDS, however the decrease of the drug release for IPEC 75/25 (ASD) is higher.

TABLE 6
Comparison of drug release regarding S-SNEDDS and ASD at the time of manufacture and
after 3 and 6 months of storage incorporating the drug substance fenofibrate

| | drug release | drug release (after 3 | drug release (after 6 |
|---|---|---|---|
| sample name | [%] | months) [%] | months) [%] |
| IPEC 75/25 AIO | 25.3 | N/D* | N/D* |
| SNEDDS | | | |
| IPEC 75/25 | 21.4 | 22.2 | 19.8 |
| SNEDDS | | | |
| IPEC 75/25 (ASD) | 5.7 | 5.3 | 1.0 |
| EUDRAGIT ® E PO | 7.0 | 6.3 | N/D* |
| (ASD) | | | |

*N/D = not determined

### Stability Studies

### Appearance

After 3 and 6 months of storage under defined and constant conditions (30° C./65% RH), the tested samples IPEC 75/25 SNEDDS, IPEC 75/25 and EUDRAGIT® E PO (only tested after 3 months), all containing the drug substance fenofibrate, did not show any notable formation of agglomerates and could be refluffed easily with the exception of EUDRAGIT® E PO. The EUDRAGIT® E PO sample could not be refluffed easily and demonstrated larger agglomerates sticking together.

The stability data regarding the dissolution study as well the thermal characterization was already presented in Tables 5 and 6.

### Fourier Transform-Infrared (FT-IR) Spectroscopy

The FT-IR measurements were performed in order to demonstrate the formation of an Interpolyelectrolytecomplex (IPEC) by coextrusion of EUDRAGIT® E PO and EUDRAGIT® L 100-55 based on structural differences of IPEC 70/30, IPEC 75/25 and IPEC 80/20 in comparison to its single copolymers (EUDRAGIT® E PO and EUDRAGIT® L 100-55) as well as an identical percentage, unprocessed powder mixture of EUDRAGIT® E PO and EUDRAGIT® L 100-55. The FT-IR spectrograms of especially IPEC 70/30 and IPEC 75/25 revealed that the characteristic spikes of the n,n-dimethylaminoethyl function of the single EUDRAGIT® L 100-55 in the wave number range of 2850-2750 cm⁻¹ were substantially diminished. Furthermore, in the wave number range of 1580 1530 cm⁻¹, spikes which indicated the presence of a carboxylate function were detected that additionally substantiated the indication of generating an IPEC by HME processing. Indications of common degradation products of methacrylates caused by the HME process (methacrylic anhydride spikes at 1805 cm⁻¹ and 1760 cm⁻¹ and n,n-dimethylaminoethanol predicted spikes at 1460 cm⁻¹ and 1040 cm⁻¹) could not be identified in the spectrograms.

### Elementary Analysis of Nitrogen Content

TABLE 7
Nitrogen content of IPEC 75/25 in comparison to its physical powder
mixture of EUDRAGIT ® E PO and EUDRAGIT ® L 100-55 (75/25)

| sample name | nitrogen content [%] |
|---|---|
| IPEC 75/25 | 3.1 |
| Powder mixture EUDRAGIT ® E PO | 3.1 |
| and EUDRAGIT ® L 100-55 (75/25) | |

The elementary analysis regarding the nitrogen content of IPEC 75/25 and its physical powder mixture comprising the same percentage of EUDRAGIT® E PO and EUDRAGIT® L 100-55 as used in IPEC 75/25 revealed a similar nitrogen content. The results may lead to the fact that no volatile nitrogenous degradation products were generated in IPEC 75/25 by the HME processing.

1: A method of preparing a solid self-nanoemulsifying drug delivery system, the method comprising:
providing a self-nanoemulsifying drug delivery system by mixing
(i) at least one pharmaceutically active ingredient;
(ii) at least one lipid component;
(iii) at least one surfactant;
(iv) optionally, at least one solvent; and
(v) optionally, at least one additive; and then
applying the obtained self-nanoemulsifying drug delivery system on a mixture comprising
(ia) 60 to 90 parts by weight of at least one dimethylaminoethyl methacrylate-butyl methacrylate-methyl methacrylate copolymer;
(iia) 10 to 40 parts by weight of at least one methacrylic acid-ethyl acrylate copolymer; and
(iiia) optionally, at least one additive;
wherein the sum of (ia) and (iia) is 100 parts by weight;
by hot melt extrusion, spray drying, adsorption, electrospinning, electrospraying, prilling by vibration, granulation or supercritical fluidization, to obtain the solid self-nanoemulsifying drug delivery system.

2: The method according to claim 1, wherein (ia) and (iia) are present in a pre-mixed powder form.

3: The method according to claim 1, wherein (ia) and (iia) and optionally (iiia) are coextruded before being applied as the mixture.

4: The method according to claim 1, wherein the at least one dimethylaminoethyl methacrylate-butyl methacrylate-methyl methacrylate copolymer
i) is obtained by radically polymerizing monomers dimethylaminoethyl methacrylate, butyl methacryalte, and methyl methacrylate in a ratio of
a) 30 to 70 wt.-% dimethylaminoethyl methacrylate;
b) 15 to 35 wt % butyl methacrylate; and
c) 15 to 35 wt.-% methyl methacrylate;
whereby a sum of a) to c) is 100 wt.-%; optionally in the presence of further additives; and/or
ii) has a residual monomer content of not more than 0.5% for each monomer; and/or
iii) has a weight average molecular weight M_{w} of 15,000 to 300,000 g/mol; and/or
iv) is obtained by a solution polymerization process.

5: The method according to claim 1, wherein the at least one methacrylic acid-ethyl acrylate copolymer
i) is obtained by radically polymerizing monomers methacrylic acid and ethyl acrylate in a ratio of
a) 35 to 60 wt.-% methacrylic acid; and
b) 40 to 65 wt.-% ethyl acrylate;
whereby a sum of a) and b) is 100 wt.-%, optionally in the presence of further additives; and/or
ii) has a weight average molecular weight M_{w} of 15,000 to 800,000 g/mol; and/or
iii) is obtained by an emulsion polymerization process with an optional subsequent drying step; and/or
iv) has a residual monomer content of not more than 0.5%, based on a sum of all monomers.

6: The method according to claim 1, wherein the at least one pharmaceutically active ingredient has a solubility of less than 0.1 mg in ml w ater at 37° C., and/or is selected from the group consisting of celecoxib, efavirenz, fenofibrate, and mixtures thereof.

7: The method according to claim 1, wherein the at least one lipid component is selected from the group consisting of C₆-C₁₂ fatty acid triglycerides; C₁₃-C₂₁ fatty acid triglycerides; propylene glycol dicaprylate/dicaprate; glyceryl tricaprylate/tricaprate; glyceryl triricinoleate; lauric acid triglycerides; glyceryl dibehenate; linoleic acid and oleic acid triglycerides; linoleic acid, oleic acid, and palmitic acid triglycerides; ethyl oleate; isopropyl myristate; monolinoleate triglycerides/diglycerides/monoglycerides; glyceryl tricaprylate/tricaprate/trilaurate; oleic acid; oleic acid and palmitic acid triglycerides; palmitic acid, oleic acid, and linoleic acid triglycerides; oleic acid, linoleic acid, and palmitic acid triglycerides; linoleic acid, oleic acid, and palmitic acid triglycerides; linoleic acid, oleic acid, alpha-linolenic acid, and palmitic acid triglycerides; linoleic acid, oleic acid, and stearic acid triglyceride; glyceryl triacetate; glyceryl tricaprylate; hard fat; and of any mixtures thereof.

8: The method according to claim 1, wherein the at least one surfactant is selected from the group consisting of poly oxy ethylene (23) lauryl ether; poly oxyethylene (2) oleyl ether; glyceryl monooleate; caprylate and caprate monoglycerides/diglycerides; glyceryl monocaprylate; propylene glycol monocaprylate; polyoxyl-35 hydrogenated castor oil; polyoxyl-40 hydrogenated castor oil; lauroyl polyoxyl-32 glycerides; stearoyl polyoxyl-32 glycerides; polyoxyl-15 bydroxystearate; triblock copolymer of polyoxyethylene and polyoxypropylene; oleoyl polyoxyl-6 glycerides; linoleoyl polyoxyl-6 glycerides; lauroyl polyoxyl-6 glycerides; caprylocaproyl polyoxyl-8 glycerides; propylene glycol monolaurate; polyoxyl-40 stearate; diacetylated monoglyceride; polyglyceryl-3 dioleate; sorbitan monolaurate; sorbitan monooleate; sorbitan sesquioleate; sorbitan trioleate; glyceryl monostearate; d-α-tocopherol polyethylene glycol 1000 succinate; polyoxyethylene sorbitan monolaurate; polyoxyethylene sorbitan monostearate; mid polyoxyethylene sorbitan monooleate; and any mixtures thereof.

9: The method according to claim 1, wherein the at least one solvent is selected from the group consisting of diethylene glycol monoethyl ether, polyethylene glycol 200, polyethylene glycol 400, polyethylene glycol 6000, propane-1,2,3-triol, (z)-octadec-9-enylamine, polypropylene glycol, propylene glycol, 2-pyrrolidone, tetraethylene glycol, diethylene glycol monoethyl ether, and any mixtures thereof.

10: The method according to claim 1, wherein the at least one additive is selected from the group consisting of antiadherents; binders; flavors; pigments; disintegrants; glidants; flow regulators; antioxidants; sweeteners; antistatics; and mixtures thereof.

11: The method according to claim 1, wherein the self-nanoemulsifying drug delivery-system is present in 1 to 30 wt-% based on a total weight of the self-nanoemulsifying drug delivery system and the mixture.

12: The method according to claim 1, wherein
(i) is present in 0.1 to 15 wt.-%;
(ii) is present in 5 to 40 wt.-%;
(iii) is present in 5 to 60 wt.-%;
(iv) is present in 10 to 50 wt.-%; and
(v) is present in 0 to 25 wt.-%;
based on a total weight of the self-nanoemulsifying drug delivery system.

13: Solid A solid self-nanoemulsifying drug delivery system, obtained by the method of claim 1.

14: The solid self-nanoemulsifying drug deliver system according to claim 13, wherein the solid self-nanoemulsifying drug delivery system is a nutraceutical product or a medicament.

15: The solid self-nanoemulsifying drug delivery system according to claim 13, for use as a medicament.
